(19) United States
(12) Patent Application Publication
(10) Pub. No.: US 2017/0031440 A1
(43) Pub. Date: Feb. 2, 2017

(54) BRAIN-CONTROLLED INTERFACE SYSTEM AND CANDIDATE OPTIMIZATION FOR SAME

(71) Applicant: Adriane B. Randolph, Atlanta, GA (US)

(72) Inventor: Adriane B. Randolph, Atlanta, GA (US)

(73) Assignee: Kennesaw State University Research and Service Foundation, Inc., Kennesaw, GA (US)

(21) Appl. No.: 15/215,868

(22) Filed: Jul. 21, 2016

Related U.S. Application Data
(60) Provisional application No. 62/197,812, filed on Jul. 28, 2015.

(51) Int. Cl. G06F 3/01; A61F 4/00; A61B 5/00; A61B 5/1455; H04W 84/18; A61B 5/0482

(52) U.S. Cl. CPC G06F 3/015; H04W 84/18; A61B 5/0482; A61B 5/6803; A61B 5/742; A61B 5/14553; A61F 4/00

(57) ABSTRACT

A system and method for using the system that permits a severely-ability challenged user to communicate with his or her environment using a brain-controlled interface. In one embodiment, the user's functional capability with the system is assessed to enhance candidate optimization for system benefit.

This application claims the benefit of U.S. Provisional Application No. 62/197,812, filed Jul. 28, 2015, which is incorporated by reference herein in its entirety.

A system permitting function using a brain-controlled interface and candidate optimization for system benefit.

Disability classification includes sensory, physical, mental, self-care, difficulty going outside the home, and employment disability. Motor disabilities that limit desired activities can be circumvented using brain-controlled interfaces. Individuals with severe motor disabilities, either temporary or permanent and either due to injury, pathology, environment (astronauts or pilots subject to severe gravitational forces), etc., may be “locked-in” their bodies, losing all voluntary movement and speech. Such individuals, while lacking unaided control of their environment, are generally cognitively intact. Input devices may assist in daily function.

### BRIEF DESCRIPTION OF THE DRAWINGS

FIG. 1 illustrates input devices and use along a continuum of motor disabilities depending on user functional capabilities.

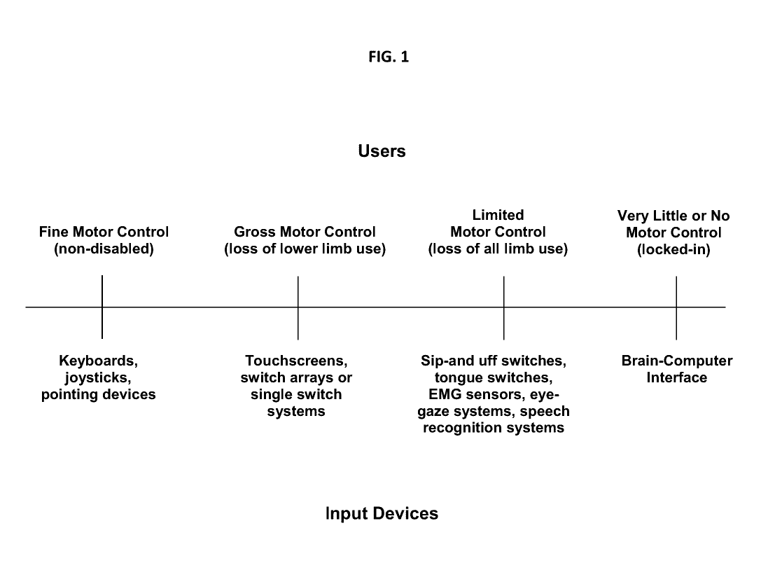

FIG. 2 shows an example brain computer interface (BCI) as an objective measure of user performance by directly measuring and recording a user's electrophysiological and metabolic outputs for basic components of interaction.

FIG. 3 schematically shows individual-technology fit (ITF) model linking individual characteristics with BCI features to predict human performance, using a fit as matching perspective.

FIG. 4 shows a schematic NeuroGlass design.

### INPUT DEVICES

Assistive technologies (AT) in general augment the functional capabilities of individuals with and without disabilities and, in traditional form, use voluntary movements for input, e.g., manipulating a mouse or joystick, a sip-and-puff head switch, etc. In non-traditional form, AT uses neurophysiological measures for non-muscularly controlled computer applications.

Brain-computer interfaces (BCI) significantly benefit individuals with severe motor disabilities to improve their quality of life when paired with the inventive system. Compared to traditional devices based on direct physical movement, BCIs record electrophysiological and metabolic signals. BCIs vary according to the type of neurophysiological signal recorded, method used for recording, and cognitive tasks employed. Most applications target disabled users who are cognitively intact but have such severely limited mobility that system input through physical movement using a keyboard, mouse, joystick, head switch, or eye-gaze device is not feasible. BCIs thus provide non-traditional assistance for controlling computers using neural input.

BCI often have high error rates and low information transfer rates or bandwidth. Efficacy may be limited by both an individual's ability to provide distinguishable changes in their neurophysiological input, and by the usability of the control interface provided to the individual.

A system's usability is enhanced when it is adjustable to meet the needs of specific individual users, such as in traditional AT. However, for non-traditional AT such as BCI, a gap still remains because there is currently no formalized process for determining a user's aptitude or literacy for control of various BCIs without testing on an actual system. Current methods to accommodate individuals are through trial-and-error, consuming substantial time and resources in an effort to determine applicability and fit.

In one embodiment, the inventive system encompasses an individual-technology fit (ITF) model and method for BCI. The results are obtained from an objective assessment of an individual's ability to control a specific BCI, using both able-bodied and the targeted disabled end-user individuals, and based on continuous analysis of inputs from the brain recorded as electroencephalograms (EEG). The system uses a comprehensive, user-centered approach to match an individual's characteristics to the characteristics of a particular BCI to optimize system performance. Using quantitative methods with data from targeted end-users allows non-traditional AT to be selected and adapted based upon individual user characteristics. The results permit enhanced pairing of individuals suffering from “locked-in syndrome” and other motor control disabilities with devices that improve their quality of life and rehabilitation efforts.

While BCIs provide users with the ability to communicate with and control environmental, navigational, and prosthetic devices, their efficacy is limited by the user's ability to provide distinguishable changes in their neurophysiological input. Such BCI literacy and usability of the control interface are affected by various factors, e.g., current fatigue level and physiology. Informational science can inform neuroscience by examining the match between an individual user and BCI technology, resulting in an individual-technology fit (ITF). BCI technologies are often matched to users through trial-and-error processes. These processes increase the level of BCI illiteracy and wastes time and resources.

A methodology that ties performance to available BCI technologies based on individual characteristics, such as demographic, physiologic, and cognitive traits, may expedite the technology-fit process. Because individuals vary in their characteristics, ITF requires developing paradigms and heuristics that link individual characteristics to available technologies to determine the most effective approach, improving the time and resources expended in offering impactful solutions to a sensitive user population.

The inventive method provides non-invasive techniques for recording BCI input. Surface-mount sensors, versus sensors that are surgically implanted, are placed on surface of the skin for signal acquisition. Electroencephalography (EEG), a bio-recording technique to measure electrical activity of the brain, is a common example of information obtained from electrodes placed on surface of the scalp. Other examples include galvanic skin response (GSR) as a psychophysiological input for non-muscle control of a computer interface, and functional magnetic resonance imaging (fMRI) and functional near-infrared imaging (fNIR) as non-invasive measurements of oxygenated blood volume using, respectively, a powerful magnetized probe and near-infrared light reflection of brain activity.

Each BCI system is based on a transducer that translates electrophysiological or metabolic signals, such as human brain signals, into control signals. For example, EEG may measure a brain signal known as the mu rhythm based on activity in the brain's motor cortex region which varies according to real and imagined movement. Other such signals include slow cortical potentials, P300 and other event-related potentials (ERPs), steady state visually-evoked potentials (SSVEPs), and beta rhythms. P300 is an evoked response that occurs over the parietal region of the brain 300 ms after a stimulus. The change can be seen and measured. P300 has been used to control, e.g., a speller, interactions in a virtual world, wheelchair navigation, and a robotic arm. Properly filtered (detect/non-detect) and translated (e.g., detection converted into a directional indicator, button click, keystroke, input API call, or message), signals are output as machine-readable commands to interface with an application or control a device, e.g., to move a cursor on a screen and make selections within a user interface. Mu rhythm-based BCI may take advantage of the difference in signal properties between idle and active imagery within the brain motor cortex region to produce a continuous control signal. The proportional difference in signal properties may be measured by a response R-squared value and indicate signal strength or the degree of modulation a person may induce by either attempting or even imagining movement.

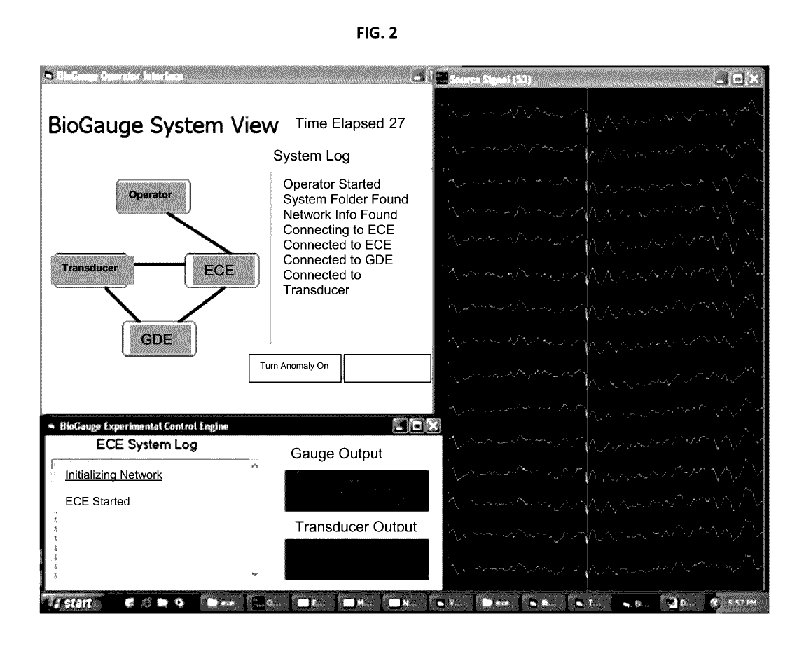

Transducer detection technology falls into three general categories: discrete, continuous, and spatial reference. The signal detection technology, the user's abilities and state, and environmental influences on the user govern transducer selection. A user's control is either no-control or intentional-control. Environmental influences include temperature, lighting, positional comfort, and electrical noise. A transducer outputs a control signal that can be analyzed at its most basic level prior to systematic adaption. Each BCI transducer can include a “BioGauges” methodology and toolset as an objective measure of user performance. BioGauges, schematically shown in FIG. 2, are simple control interfaces that directly measure and record users' electrophysiological and metabolic outputs for the basic components of interaction. BioGauges can be used to determine the range, spatial accuracy, temporal accuracy, and granularity of control for a user and a particular transducer configuration—these constitute a person's ability to control a particular transducer, termed controllability. Controllability information is used to select a device with which a user achieves his/her best performance, to better configure a BCI system for a user, or to more objectively assess the potential of a BCI technology for control. The BioGauges toolset may be implemented in a configurable architecture that consists of an operator interface, experimental control engine (ECE), a set of BioGauges contained in a Gauge Display Engine (GDE), and a BCI transducer. FIG. 2 illustrates what the operator sees when these components are all communicating with one another and responding to user EEG input.

BioGauges characterize controllability of discrete and continuous transducers during periods of intentional user control by their neurophysiological input such as when they wish to make a selection, and unintentional user control such as when they are idly looking at the screen reviewing content. For cases with a continuous transducer and intentional control, the individual may be presented with a bar and a cursor that he/she needs to move along the bar to hit a designated target area.

The invention will be further appreciated with respect to the following illustrative and non-limiting examples.

### Example 1

A discrete EEG-based transducer was tested with five able-bodied individuals. With the low-frequency asynchronous switch design (LF-ASD) that provides a single-trial switch based on differences between active and idle states, individuals achieved more than 73% accuracy for reaction time, 96% for temporal accuracy, and 82% with repeated accuracy.

### Example 2

A continuous GSR-based transducer was tested with six able-bodied individuals. The results demonstrated that untrained individuals were able to exhibit stability and control at more than 87% accuracy, but BioGauges demonstrated that it was difficult for individuals to hold their GSR signal at heightened arbitrary levels for prolonged periods or to hold plateaus of excitement due to mental exhaustion after tasks that required higher excitement levels. One individual noted a headache that developed during the test sessions.

### Example 3

Ten able-bodied individuals were tested to compare mu rhythm-based control to GSR control. There was little variation in participant control with either transducer because adaptive algorithms were not utilized. Further, due to low strength of the raw mu values, all participants were able to obtain targets in one direction but not the other. Use of adaptive algorithms can increase the sensitivity and responsiveness of the BCI to small changes in mu.

### Example 4

Thirty-three able-bodied individuals and five amyotrophic lateral sclerosis (ALS) individuals were tested to compare fNIR control to GSR control. Participants were able to exhibit some level of control of both the fNIR and GSR technologies. Seventy-four percent of all participants, and 60% of ALS participants, achieved greater than chance results with fNIR. For GSR, 60% of all participants and 40% of ALS participants achieved greater than chance results.

BioGauges have shown that individuals may have better literacy with one type of BCI transducer over another.

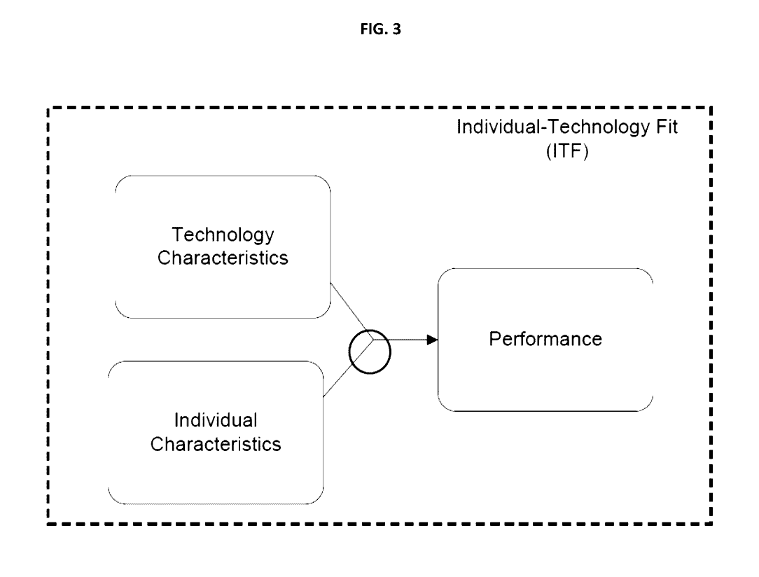

In the context of BCI, the concept of fit, used to describe contingent relationships between variables, may be classified according to moderation, mediation, matching, gestalts, profile deviation, and covariation. Fit as moderation, mediation, and matching specify a relationship typically between just two variables; fit as gestalts, profile deviation, and covariation specify a relationship typically among multiple variables. Individual-technology fit (ITF), FIG. 3, is the extent to which individual characteristics match with technology features to enable an individual's control of a technology such as BCI. FIG. 3 shows an ITF model linking individual characteristics with BCI features to predict human performance, using a fit as matching perspective.

This ITF model does not include utilization because utilization of BCI technology may be considered mandatory for certain users if they have no other alternatives and a strong desire for communication and control. Further, utilization considers ongoing use and this ITF model is for explaining initial performance. Task can be excluded if it is held constant, e.g., a BioGage-like task, in an effort to focus on the impact of individual characteristics with technology features. In other models, tasks may be included in order to improve overall quality of life versus performance in any specific testing or real-world task.

The technology characteristics of BCI transducers may be described by the following transducer taxonomy and attributes:

1. Type classifies the general response mechanism used, i.e., endogenous, exogenous, or modulated response. An endogenous type of transducer is based on internally generated user control. An exogenous type of transducer is based on an automated response to external stimuli. A modulated response type of transducer is based on an individual's internally modulated response to external stimulation.

2. Biorecording technology is used to record participant signals, e.g., EEG, fNIR, fMRI, GSR.

3. Inputs classify sensor or electrode placement, e.g., areas over the brain or selected muscle groups such as in the fingers.

4. Neurological phenomena control the transducer, and can be phenomena in electrical brain activity, in blood oxygenation, or in skin conductance.

5. Inclusion of a stimulator which can, if applicable, provide a stimulus to cue exogenous transducers.

6. Feature extraction/translation algorithms which extract and translate the signal into a useful control signal.

7. Type of transducer output, i.e., discrete, continuous, or spatial reference. Discrete transducers produce output in a set of states such as a switch; continuous transducers produce an ongoing stream of output within a range; and spatial reference transducers produce output in a particular point in 2- or 3-dimensional space that can be selected.

8. Idle support indicates whether the transducer supports a state where the user is not intending to control the technology, i.e., No Control State.

Individual distinguishing characteristics include demographic, physiological, and cognitive differences. Individual characteristics that best match with particular BCI technologies range from functional limitations to the amount of system training received. Applicants examined a set of 28 characteristics potentially affecting BCI control and tested them with fNIR- and GSR-based technologies. Characteristics ranging from age and athleticism to hair color were selected based on a review of related literature and discussions with researchers in the fields of BCI and AT concerning observed and hypothetical physiological effects. The results demonstrated that age, regular caffeine consumption, and education level positively correlated with fNIR control, whereas age, sex, hair and skin color, hair texture, meditation, regular alcohol consumption, and video game experience positively correlated with GSR control. The interaction of age and hand-and-arm movement predicted modulation of the mu rhythm in a mu-based BCI, with a history of playing at least one instrument, not being on affective drugs, being female, and being over the age of 25 positively correlated with mu rhythm modulation.

Performance is the observable evidence of fit between individual characteristics and a particular BCI technology. Performance should increase as individuals are well matched to BCI, and performance should decrease as individuals are not well matched. Performance may be measured by time consumed and/or accuracy in executing a specific testing task, such as the horizontal bar and a cursor test mentioned previously, related tests of ability to move a cursor, pointer, or other selection element to a defined region of a display, or ability to make selections after receiving a prompt, e.g., a simplified, simulated speller-like task. Performance may also be measured by time consumed and/or accuracy in performing a suite of one or more real-world tasks, such as controlling a speller application, a user interface in a graphical display, user interfaces to various other applications such as controllers for electromechanical devices, etc.

Systems usable for ITF modeling or as non-traditional AT systems typically include at least one transducer, a biosignal amplifier, an analog-to-digital converter, a digital signal processor, and a BCI software interface that is output to a display. The transducer, as described above, may be an EEG transducer, a GSR transducer, an fNIR transducer (illuminator/photodetector combination), or, generally for ITF modeling, comparison, and research, an fMRI imager. For investigatory applications, such systems may include one or more different types of BCI transducers. BCI transducers may be individually and separately disposed on an individual, e.g., using self-adhesive patches, or may be disposed on the individual in a pre-manufactured array, such as in known cranial EEG nets and headcaps or on other structures such as helmets, frames for glasses or goggles, or a head-mounted display. The various transducers are operably connected to the biosignal amplifier by a wiring harness, with the biosignal amplifier functioning to normalize the signal measured by the transducer, e.g., a voltage signal for EEG, current for photodetected light in fNIR, conductivity for GSR, etc., and amplify it for digital sampling. Examples of device suited for use in investigatory settings include integrated biosignal amplifiers such as a Gugertec g.USBamp or BioSemi ActiveTwo Bioamplifier, units that provide both signal amplification and analog-to-digital conversion for digital output to a digital computer. Otherwise, an analog-to-digital converter is operably connected to the biosignal amplifier by one or more sampling channels, typically one per transducer (minus any reference transducers), to sample signals from the transducers when disposed at various predetermined locations on the individual. The analog-to-digital converter (or converter stage of an integrated biosignal amplifier) communicates the sampled signals to the digital signal processor.

In such investigatory systems, the digital signal processor is usually a general purpose digital computer executing a data analytics application. Such applications include online applications such as LabVIEW, offline applications such as MATLAB, and specialized BCI software such as BCI2000 (available at http://www.schalklab.org/research/bci2000) which process the sampled signals using a classifier. Depending upon the type(s) of transducer employed, the classifier may distinguish from the signal, e.g., intentional control events versus unintentional events as reflected in mu rhythm signal modulation, a decision-making event as reflected in P300 potentials, localized or global brain activity triggered by motor imagery, fNIR detection of increased oxygenation, and varying levels of arousal indicated by minute differences in generated sweat. For example, EEG transducers may be used to detect mu rhythm modulation in the sensorimotor cortex induced via attempted or imagined movement of a limb. For further example, EEG transducers may be used to detect P300 potential in the parietal lobe induced by a decision that one of a number of sequentially-highlighted options is both currently highlighted and an intended selection. Examples of classifier operation, configuration, and theory for mu signal modulation and P300 potential detection are published at http://www.bci2000.org/wiki/index.php/User_Tutorial:Analyzing_the_lnitial_Mu_Rhythm_Session and http://www.bci2000.org/wiki/index.php/User_Tutorial:Obtaining_P300_Parameters_in_a_Calibration_Sess ion. Where the digital signal processor is a general purpose computer, the same digital signal processor may use the output of the classifier as the input for a BCI software interface such as a so-called “speller” application. For example, BCI2000 includes a P300 potential-driven speller module similar to that first disclosed by Farwell and Donchin in 1988. The output of the application is provided to a display, such as the screen of a laptop or an external monitor, and the individual controls operation of the application through the BCI interface. For example, the individual may visualize motor movement of a limb on one side of the body to direct a cursor or pointer in the corresponding direction, a selection box to the previous selection, etc. The individual may visualize motor movement of another limb on the same side of the body to direct the cursor or pointer up or down, to shift a selection box to a different group of selections etc. The individual may visualize motor movement of opposing limbs simultaneously to select an item under a cursor or pointer, the current selection box selection, etc. Variations where other muscle groups are used, such as the fingers, toes, and tongue, are apparent to those skilled in the art. Variations where sequences of motor movement or muscle groups are used to trigger actions such a selection, deletion, application switching, etc. will also be apparent.

The translation of classified signals by the digital signal processor may alternately be output to a separate BCI software interface as a simulated directional indicator, button click, keystroke, input API call, or message. For example, the BCI software interface might simply be a standard device interface (e.g., mouse or joystick) provided with directional indicators and simulated button clicks output by the signal processor. Alternately, the BCI software interface may be an AT software application which exchanges remote API calls or messages with the digital signal processor, displaying interfaces such as a speller application for communication with others via text, an assistive device control application for the operation of motorized chairs, beds, and the like, and an environmental control application for the operation of lights, fans, life support systems providing food and/or hydration, etc. The latter, in particular, may be controlled using Low-Frequency Asynchronous Switch Device-like logic by individuals constrained to low-data rate communication (especially those using fNIR-based BCIs).

Investigatory systems are useful for flexibly determining technology characteristics, such as the effects of different BCI transducers and even biosignal amplifiers upon the ability of BCI systems to detect control efforts, the ability of different classification approaches/algorithms to accurately classify intentional-control and no-control states and, if used in a covariation analysis, relationships between technology characteristics and task as part of an ITF evaluation. However, in non-investigatory use such flexibility may instead constitute a significant disadvantage.

In personal use systems, an all-in-one or lightweight and mobile device may improve real-world function for communication and environmental control by individuals with severe motor disabilities and intact cognition. Limited space in an individual's home environment and difficulty maintaining a display unit within the user's field of view either when the individual is mobile (in the case of so-called external displays) or when a caregiver is assisting the individual (in the case of an integral laptop display for a laptop mounted to a wheelchair, medical/transport bed, or other transport device). After evaluation of an ITF, flexibility in the selection of BCI transducers, amplification and digitization technology and classification should no longer be required. Instead, an all-in-one or closely-coupled AT device should be used.

An improved personal use system may similarly include at least one transducer, a biosignal amplifier, an analog-to-digital converter, a digital signal processor, and a BCI software interface that is output to a display. However, a predominant number of these components, particularly those involved in signal acquisition, are co-located within a head-mounted display device (HMDD) to be worn by the individual. In contrast to exiting EEG nets or headcaps, the device incorporates one or more of (1) an in-ear BCI transducer, (2) a circumaural BCI transducer (including so-called over-the-ear or behind-the-ear positioning), or (3) a cranial transducer disposed on a device-attached extension arm. The HMDD may be a superimposed display device permitting observation of the surrounding environment, such as a Google Glass-like HMDD, or less desirably an obscuring display device of the sort used for virtual reality environments, such as an Oculus Rift-like HMDD. With such devices, the BCI transducer or transducers may be included as earbud-like accessories operably connected to an arm or “temple” of the device frame, as integrated components of the arm or “temple” (particularly the so-called “temple tip”), or as integrated components of an extension arm or headband affixed to the arm. A biosignal amplifier and analog-to-digital converter may be housed within the HMDD, and connective wiring run through the arm, extension arm, and/or headband to minimize exposed wiring.

In one embodiment, the digital signal processor may be co-located and housed within the HMDD. To reduce size and power, the digital signal processor may be a specialized digital signal processor coupled to a low power general purpose system on chip (SoC). Alternately, the digital signal processor may be an advanced SoC including digital signal processor (DSP) and/or single instruction multiple data (SIMD) architectural extensions. Such systems may execute a classifier appropriate for the associated BCI transducer, as discussed above, as well as a BCI software interface such as a speller, on-screen keyboard, cursor or pointer, a selection box used to select or toggle menu entries, check boxes, and other similar GUI element, etc., as well as specialized applications such as for assistive device, environmental, and life support control. Display output from the SoC would be displayed on the HMDD with the variation providing a so-called “all-in-one” BCI AT device. Display from the SoC may also be optionally mirrored to other external displays, including monitors, smartphones, tabled displays, laptops, etc. for communication to caregivers or others.

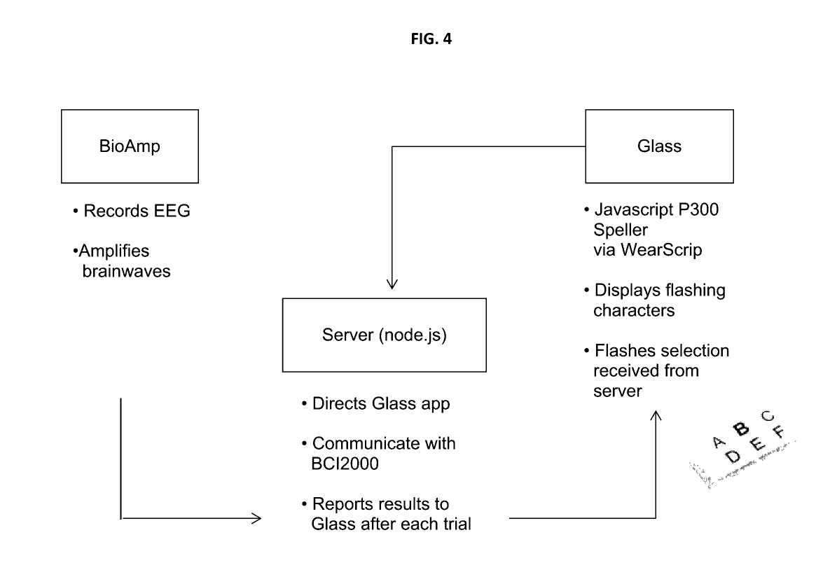

In one embodiment, digital signal processing may be offloaded to a companion mobile device such as a smartphone, tablet, or laptop. In this embodiment, the analog-to-digital converter may output to a wired or low power wireless interface co-located and housed within the HMDD, such as Apple's Lightning interface, USB, Bluetooth or Wi-Fi, with the companion mobile device functioning as the digital signal processor via network connection as with a UDP port. Depending on the capabilities of the HMDD, i.e., whether the display is an autonomous or semi-autonomous device that executes its own applications, such as Google Glass, or is a display peripheral necessarily coupled to a video output of a companion device, output from the companion device may be sent to the HMDD in the form of an input to a BCI software interface executed by the HMDD (e.g., an output of the classifier or an output of cursor, pointer, and/or selection box control commands based upon the output of the classifier) or an image of a BCI software interface and other applications being executed by the companion mobile device (e.g., a screen projection of the companion mobile device BCI software interface and other applications over wireless display (WiDi), virtual network console (VNC), or so-called remote desktop (RD) interfaces). For example, in a system using Google Glass as an HMDD, the companion mobile device may communicate with an Android-based Glassware speller application executing on the Glass device to provide the classifier output to the HMDD-executed BCI software interface. Alternately, the companion mobile device may at least partially execute the BCI software interface and direct the Glass device to display visible elements of a speller user interface via a framework such as the web-based, javascript-driven WearScript framework developed for glass. For further example, in a system using an Oculus-Rift like HMDD, the companion mobile device may execute the BCI software interface and output its display to the HMDD display screen(s). A schematic NeuroGlass design is shown in FIG. 4.

The selection of BCI transducers, the physical configuration of the HMDD device, and the processing applied by individual personal-use systems (e.g., mu rhythm signal modulation, P300 potential detection, fNIR activity detection, and GSR response) in such a device may be determined by the individual's ITF. The selection of appropriate classifier for executing by the digital signal processor and BCI software interface for execution by the system would follow from such selections, as well as transducer placements optimized through less extensive individualized testing on actual systems.

The disclosed inventive method and system contributes to a shift from a “one-size-fits-all” healthcare model to a more individualized, subject-specific model and may serve as a foundation for all use of BCIs. Evaluation of technology characteristics, individual characteristics and, optionally, task characteristics enables selection of BCI transducer technologies and configurations suitable for use by an individual with limited mobility. The disclosed personal use devices enhance assistive flexibility and quality of life by eliminating the need for an individual to be moved to fixed location (e.g., desk with biosignal amplifier, digital signal processor/computer, and external display) or burdened with bulky, chair- or bed-mounted systems.

The embodiments shown and described in the specification are only specific embodiments of inventors who are skilled in the art and are not limiting in any way. Therefore, various changes, modifications, or alterations to those embodiments may be made without departing from the spirit of the invention in the scope of the following claims. The references cited are expressly incorporated by reference herein in their entirety.

What is claimed is:

1. A method and system for neural input to a mainstream device using individual technology fit of at least one brain-computer interface resulting in neural control and communication.

2. The method and system of claim 1 comprising a processor where the processor is sufficiently lightweight for mobility.

3. The method and system of claim 1 further comprising personalized display settings.

4. The method and system of claim 1 supportable from a caregiver by a mobile device.

5. The method and system of claim 1 performed using glassware.

6. The method of claim 5 where the glassware is existing.

7. The method of claim 5 where the glassware is created.

8. The method and system of claim 1 that is scalable.

9. The method and system of claim 1 involving P300 evoked potential.

10. The method and system of claim 1 involving mu evoked potential.
